(19) United States
(12) Patent Application Publication
(10) Pub. No.: US 2016/0311746 A1
(43) Pub. Date: Oct. 27, 2016

(54) METHOD OF PRODUCING ADIPIC ACID OR AT LEAST A RESULTANT PRODUCT THEREOF

(71) Applicant: BASF SE, Ludwigshafen (DE)

(72) Inventors: Rolf PINKOS, Bad Dürkheim (DE); Martin BOCK, Ludwigshafen (DE)

(21) Appl. No.: 15/103,158

(22) PCT Filed: Dec. 12, 2014
(86) PCT No.: PCT/EP2014/077598

§ 371 Date: Jun. 9, 2016

(30) Foreign Application Priority Data
Dec. 13, 2013 (EP) 13197159.0

(51) Int. Cl. C07C 51/36; C07C 29/149; C07C 209/16; C08G 69/28

(52) U.S. Cl. CPC C07C 51/36; C08G 69/28; C07C 29/149; C07C 209/16

(57) ABSTRACT

The present invention relates to a process for preparing adipic acid or at least one conversion product thereof, in which muconic acid is hydrogenated with hydrogen in the presence of at least one transition metal catalyst C and of an aqueous liquid A in a reaction zone, wherein the muconic acid is at least partly insoluble in the liquid A under the hydrogenation conditions.

### BACKGROUND OF THE INVENTION

The present invention relates to a process for preparing adipic acid or at least one conversion product thereof by catalytic hydrogenation of muconic acid.

### STATE OF THE ART

Adipic acid is an industrially important commodity and is used particularly for preparation of polyamide-6,6, which is also referred to as nylon. The preparation of polyamide-6,6 by water-eliminating polycondensation of adipic acid with hexamethylenediamine has long been known. The adipic acid used in the preparation of polyamide-6,6 is prepared industrially particularly by oxidation of cyclohexanol or cyclohexanone/cyclohexanol mixtures, which are also referred to as anolone, with concentrated nitric acid. Anolone is obtainable by oxidation of cyclohexane with atmospheric oxygen.

A further known starting material for preparation of adipic acid is muconic acid. Muconic acid, systematic name hexa-2,4-dienedicarboxylic acid, may be present in cis,cis, cis,trans or trans,trans conformation. Muconic acid can be obtained, for example, by biochemical processes from renewable raw materials such as glucose or lignin. Full hydrogenation of the carbon-carbon double bonds in the muconic acid gives adipic acid.

In contrast to adipic acid, muconic acid features notable lack of solubility in standard solvents such as water and ethanol. For example, cis,cis-muconic acid at about 47° C. exhibits a solubility in water of about 1 g per 100 g of water. Therefore, the conversion of muconic acid on the industrial scale, for example to adipic acid, is generally associated with an elevated level of complexity. For this reason, the literature includes various procedures for converting muconic acid to a form of better solubility before the further conversion thereof, for example to adipic acid. These include, for example, esterification with short-chain alcohols and neutralization with bases in order to obtain soluble muconic salts.

E. H. Farmer and L. A. Hughes, J. Chem. Soc. 1934, 1929-1938, describe the preparation of adipic acid proceeding from the disodium salt of muconic acid in an aqueous medium with hydrogenation catalysts based on nickel or platinum.

J. A. Elvidge et al., J. Chem. Soc. 1950, 2235-2241, describe the preparation of cis,trans-muconic acid and the hydrogenation thereof to adipic acid in ethanol in the presence of a platinum catalyst. No details are given of the amount of solvent used and the catalyst.

U.S. Pat. No. 4,968,612 describes a fermentation process for preparation of muconic acid proceeding from toluene and the hydrogenation of the muconic acid thus obtained to adipic acid. Specifically, muconic acid is reacted as a 40% by weight slurry in acetic acid and in the presence of a palladium catalyst on charcoal. The water content of the acetic acid used is unspecified. A disadvantage of this mode of reaction is the use of corrosive acetic acid, which entails the use of high-quality corrosion-resistant reactors. A disadvantage of this process is that toluene that does not originate from renewable sources is used as the starting material.

K. M. Draths and J. W. Frost, J. Am. Chem. Soc. 1994, 116, 399-400 and W. Niu et al., Biotechnol. Prog. 2002, 18, 201-211 describe the preparation of cis,cis-muconic acid from glucose by biocatalyzed synthesis with subsequent hydrogenation of the cis,cis-muconic acid with the aid of a platinum catalyst to adipic acid. In the two cases, the pH of the fermentation mixture prior to the hydrogenation is adjusted to above 6.3, or to a value of 7.0. This results in a solution of muconic salts. Since, in the two cases, the fermentation broth is first centrifuged and only the supernatant is used for hydrogenation, and according to the procedure of Niu et al. the supernatant is additionally twice admixed with activated carbon and filtered prior to the hydrogenation, it can be assumed that the hydrogenation mixture does not comprise any solid muconic acid.

A further process for preparing muconic acid from renewable sources is described, for example, in WO 2010/148080 A2. According to example 4, in paragraphs [0065] and of this document, 15 g of cis,cis-muconic acid and 150 mL of water are heated under water reflux for 15 minutes. After cooling to room temperature, filtration and drying, 10.4 g (69%) of cis,trans-muconic acid are obtained. The mother liquor (4.2 g=28% by weight, based on cis,cis-muconic acid) no longer consists of muconic acid, It comprises lactones and further, unknown reaction products.

J. M. Thomas et al., Chem. Commun. 2003, 1126-1127, describe the hydrogenation of muconic acid to adipic acid with the aid of bimetallic nanocatalysts which have been intercalated into the pores of a mesoporous silicon dioxide by means of specific anchor groups, in pure ethanol.

WO 2010/141499 describes the oxidation of lignin to vanillic acid, the decarboxylation of the latter to 2-methoxyphenol and further conversion to catechol, and finally oxidation to muconic acid, and hydrogenation of muconic acid obtained in this way with various transition metal catalysts to adipic acid. The solvent used for the hydrogenation is unspecified.

WO 2012/170060 describes a process for preparing nitrogen compounds, especially hexamethylenediamine. The starting materials used are diammonium adipate-containing fermentation broths. In a suitable embodiment, they are produced by fermentative conversion of D-glucose to cis,cis-muconic salts. At the same time, the pH is kept below 7 by addition of ammonia. Subsequently, the cis,cis-muconate is hydrogenated at room temperature in the presence of 10% platinum on charcoal at a hydrogen pressure of 50 psi (3.4474 bar). The low temperature is necessary since, at higher temperatures, ammonia would add onto the muconic acid or salts thereof in the manner of a Michael addition. The resulting hydrogenated fermentation broth comprises diammonium adipate (DAA) with or without monoammonium adipate (MAA) and/or adipic acid (AA). A disadvantage of the process described in WO 2012/170060 is that the DAA and MAA are converted to AA prior to the further reaction, meaning that the ammonia has to be removed. This is effected by distillation in two steps, with distillation of aqueous DDA solution in the first step in such a way that ammonia and water are removed overhead. The bottom product of the distillation is cooled and the solid formed, consisting of MAA, is removed. In the second step, an aqueous MAA solution is heated with addition of water and ammonia-comprising water vapor is removed. The solid obtained after cooling consists of adipic acid. The adipic acid thus obtained is hydrogenated to hexane-1,6-diol and hexane-1,6-diol is aminated with ammonia to give hexamethylenediamine. There is no statement as to the conditions under which the hydrogenation of MAA and AA to hexanediol and the amination of hexanediol to hexamethylenediamine take place, nor as to the catalysts present therein. The sole pointer in this regard is the formula schemes in FIGS. 3, 4 and 5.

X. She et al., ChemSusChem 2011, 4, 1071-1073, describe the hydrogenation of trans,trans-muconic acid to adipic acid with rhenium catalysts on a titanium dioxide support in solvents selected from methanol, ethanol, 1-butanol, acetone, toluene and water. The results compiled in FIG. 1 of this document show the conversion and distribution of the dimethyl esters 1 (dimethyl adipate), 2 (dimethyl 2-hexenedioate) and 3 (dimethyl 2,4-hexadienedicarboxylate) obtained in the hydrogenation of trans,trans-muconic acid in methanol. The best result was achieved with an Re/TiO₂ catalyst. However, this reaction is not a process for preparing adipic acid by hydrogenating muconic acid, since ester formation proceeds as a major reaction step in every case.

The supplementary information relating to this document also describes the hydrogenation of trans,trans-muconic acid with an Re/TiO₂ catalyst at 120° C. and a hydrogen pressure of 1000 psi (68.95 bar) in water as solvent. Because of the low solubility, the muconic acid concentration in the water used as solvent was only 50 mg/mL (5% by weight). With the catalyst used, only a low selectivity based on the adipic acid is achieved in water; the main product is dihydromuconic acid. For instance, after 5 hours of reaction time, the selectivity for adipic acid was only about 10% and that for dihydromuconic acid about 90%. The authors therefore explicitly advise against the use of water as solvent for the hydrogenation reaction. Thus, the following is stated at page 4 lines 1-2 of the supplementary information: “Water is a poor reaction media toward the formation of (I) (=adipic acid).

The processes known from the prior art for preparing adipic acid from muconic acid have numerous disadvantages. If the muconic acid is used in salt form, it first has to be prepared in an additional step. In addition, the use of muconic salts leads to the unwanted occurrence of salt in the product mixture, which has to be removed in a costly and inconvenient manner. The hydrogenation of muconic acid in alcoholic solvents, for example methanol or ethanol, leads to by-products such as the monoesters and diesters of muconic acid, of dihydromuconic acid and of adipic acid with the aforementioned alcohols. This reduces the yield of adipic acid and complicates the isolation of the adipic acid. The use of corrosive solvents such as acetic acid requires expensive, corrosion-resistant reaction vessels. In many solvents, for example ethanol, only muconic acid solutions of low concentration can be obtained, and these result in low space-time yields if only these solutions and not suspensions are used for hydrogenation. In many cases, expensive catalysts, for instance based on platinum, are used. The organic solvents used are in many cases combustible and/or the use thereof is undesirable for health and environmental reasons. The yield of adipic acid in the processes described in the prior art are inadequate in many cases.

It is an object of the present invention to provide a process which enables the hydrogenation of muconic acid to adipic acid in high yield and selectivity. At the same time, the above-described disadvantages of the prior art are to be avoided. More particularly, hydrogenation should be effected, if at all possible, using muconic acid itself and not a muconic salt. Specifically, the process should be performable in a simple and inexpensive manner and in an environmentally friendly reaction medium, without the need for a costly and inconvenient removal of by-products.

It has now been found that, surprisingly, these objects are achieved by hydrogenating muconic acid at least partly in solid form with hydrogen in an aqueous liquid in the presence of a transition metal catalyst.

### SUMMARY OF THE INVENTION

The invention therefore relates to a process for preparing adipic acid or at least one conversion product thereof, selected from hexane-1,6-diol, hexamethylenediamine and polyamide-6,6, in which muconic acid is hydrogenated with hydrogen in the presence of at least one heterogeneous transition metal catalyst C and of an aqueous liquid A in a reaction zone, wherein the muconic acid is at least partly insoluble in the liquid A under the hydrogenation conditions and wherein the transition metal catalyst C includes at least one transition metal selected from Ru, Co, Rh, Ir, Ni, Fe, Pd, Pt, Cu and Au.

In a specific embodiment, the muconic acid is used for hydrogenation in the form of a suspension. In this case, the muconic acid is present as a particulate dispersed phase in the aqueous liquid.

In a further specific embodiment, the liquid A consists solely of water,

A further specific embodiment is a process for preparing adipic acid or at least one conversion product thereof, in which muconic acid is hydrogenated with hydrogen in the presence of at least one transition metal catalyst C and of an aqueous liquid A in a reaction zone, wherein the muconic acid is at least partly insoluble in the liquid A under the hydrogenation conditions, and wherein
the liquid A has a water content in the range from 95 to 100% by weight, based on the total weight of the liquid A,
the transition metal catalyst C comprises metallic nickel, metallic cobalt, metallic rhodium or a mixture of at least two of these metals, and
at least a portion of the reaction mixture is withdrawn from the reaction zone, the reaction mixture withdrawn is subjected to a separation into an adipic acid-enriched fraction and an adipic acid-depleted fraction, the adipic acid-depleted fraction is at least partly recycled into the reaction zone.

The invention further provides a process for preparing hexane-1,6-diol, in which
a) muconic acid is subjected to a hydrogenation with hydrogen in the presence of an aqueous liquid A and in the presence of at least one transition metal catalyst C as defined above and hereinafter, to obtain adipic acid,
b) the adipic acid obtained in step a) is subjected to a reaction with hydrogen in the presence of at least one hydrogenation catalyst.

The invention further provides a process for preparing hexamethylenediamine, in which
a) muconic acid is subjected to a hydrogenation with hydrogen in the presence of an aqueous liquid A and in the presence of at least one transition metal catalyst C as defined above and hereinafter, to obtain adipic acid,
b) the adipic acid obtained in step a) is subjected to a reaction with hydrogen in the presence of at least one hydrogenation catalyst to give hexane-1,6-diol,
c) the hexane-1,6-diol obtained in step b) is subjected to an amination with ammonia in the presence of an amination catalyst to obtain hexamethylenediamine.

The invention further provides a process for preparing polyamide-6,6, in which
a) muconic acid is subjected to a hydrogenation with hydrogen in the presence of an aqueous liquid A and in the presence of at least one transition metal catalyst C as defined above and hereinafter, to obtain adipic acid,
b) the adipic acid obtained in step a) is subjected to a reaction with hydrogen in the presence of at least one hydrogenation catalyst to give hexane-1,6-diol,
c) the hexane-1,6-diol obtained in step b) is subjected to an amination with ammonia in the presence of an amination catalyst to obtain hexamethylenediamine,
d) the hexamethylenediamine obtained in step c) is subjected to a polycondensation with adipic acid to obtain polyamide-6,6.

In a specific embodiment, the adipic acid used in step d) also originates at least partly from the inventive hydrogenation of muconic acid.

Specifically, in the aforementioned processes, the muconic acid used in step a) is not in salt form.

In a specific embodiment of the aforementioned processes, the hydrogenation of muconic acid is effected continuously.

### EMBODIMENTS OF THE INVENTION

Specifically, the invention encompasses the following preferred embodiments:
1. A process for preparing adipic acid or at least one conversion product thereof, in which muconic acid is hydrogenated with hydrogen in the presence of at least one transition metal catalyst C and of an aqueous liquid A in a reaction zone, wherein the muconic acid is at least partly insoluble in the liquid A under the hydrogenation conditions.
2. The process according to embodiment 1, wherein the reaction mixture, at a minimum content of 50% by weight of water, based on the total weight of the reaction mixture, has a pH at 60° C. in the range from 1 to 6, preferably 1 to 5, more preferably 1 to 4.
3. The process according to either of the preceding embodiments, wherein the liquid A has a water content in the range from 5 to 100% by weight, preferably 30 to 100% by weight, more preferably 50 to 100% by weight, particularly 65 to 100% by weight, especially 95 to 100% by weight, based on the total weight of the liquid A.
4. The process according to any of the preceding embodiments, wherein the muconic acid under the hydrogenation conditions has a solubility in the liquid A of not more than 50 g/L.
5. The process according to any of the preceding embodiments, wherein the muconic acid is at least partly in the form of particles suspended in the liquid A in the hydrogenation.
6. The process according to any of the preceding embodiments, wherein hydrogenation is effected using a liquid A in which adipic acid has a solubility under the reaction conditions of at least 100 g/L, preferably at least 200 g/L.
7. The process according to any of the preceding embodiments, wherein the muconic acid originates from renewable sources, and is preferably produced by biocatalytic synthesis from at least one renewable raw material.
8. The process according to any of the preceding embodiments, wherein the muconic acid used has a ¹⁴C-to-¹²C isotope ratio in the range from 0.5×10⁻¹² to 5×10⁻¹².
9. The process according to any of the preceding embodiments, wherein the transition metal catalyst C is a heterogeneous catalyst.
10. The process according to any of the preceding embodiments, wherein the transition metal catalyst includes at least one transition metal from groups 7, 8, 9, 10 and 11 of the Periodic Table (IUPAC), preferably selected from Re, Fe, Ru, Co, Rh, Ir, Ni, Pd, Pt, Cu and Au, particularly selected from Re, Ru, Co, Rh, Ir, Ni, especially selected from Ni, Co and Rh.
11. The process according to any of the preceding embodiments, wherein the transition metal catalyst C comprises metallic nickel, metallic cobalt, metallic rhodium or a mixture of at least two of these metals.
12. The process according to any of the preceding embodiments, wherein the transition metal catalyst C is selected from Raney nickel, Raney cobalt, rhodium on a support material, and mixtures thereof.
13. The process according to any of the preceding embodiments, wherein at least a portion of the reaction mixture is withdrawn from the reaction zone, the reaction mixture withdrawn is subjected to a separation into an adipic acid-enriched fraction and an adipic acid-depleted fraction, and the adipic acid-depleted fraction is optionally at least partly recycled into the reaction zone.
14. The process according to embodiment 13, wherein the reaction mixture is subjected to a crystallization of the adipic acid and at least a portion of the mother liquor is recycled into the reaction zone.
15. A process for preparing adipic acid or at least one conversion product thereof, in which muconic acid is hydrogenated with hydrogen in the presence of at least one transition metal catalyst C and of an aqueous liquid A in a reaction zone, wherein the muconic acid is at least partly insoluble in the liquid A under the hydrogenation conditions, and wherein
   the liquid A has a water content in the range from 95 to 100% by weight, based on the total weight of the liquid A,
   the transition metal catalyst C comprises metallic nickel, metallic cobalt, metallic rhodium or a mixture of at least two of these metals, and
   at least a portion of the reaction mixture is withdrawn from the reaction zone, the reaction mixture withdrawn is subjected to a separation into an adipic acid-enriched fraction and an adipic acid-depleted fraction, the adipic acid-depleted fraction is at least partly recycled into the reaction zone.
16. The process according to any of the preceding embodiments, wherein the hydrogenation is conducted at a temperature in the range from 20° C. to 250° C., preferably 40° C. to 150° C.
17. The process according to any of the preceding embodiments, wherein the reaction is conducted at an absolute hydrogen pressure in the range from 1 to 300 bar, preferably 2 to 100 bar.
18. The process according to any of the preceding embodiments, wherein the hydrogenation is conducted continuously.
19. The process according to any of the preceding embodiments, wherein the hydrogenation is conducted in n series-connected hydrogenation reactors, where n is an integer of at least two.
20. The process according to embodiment 19, wherein the 1st to (n-1)th reactor has a stream from the reaction zone conducted within an external circuit.
21. The process according to either of embodiment 19 and 20, wherein the hydrogenation is conducted adiabatically in the nth reactor.
22. A process for preparing hexane-1,6-diol, in which
   a) muconic acid is subjected to a hydrogenation with hydrogen in the presence of an aqueous liquid A and in the presence of at least one transition metal catalyst C as defined in any of embodiments 1 to 21, to obtain adipic acid,
   b) the adipic acid obtained in step a) is subjected to a reaction with hydrogen in the presence of at least one hydrogenation catalyst.
23. The process according to embodiment 22, wherein the hydrogenation catalyst used in step b), based on the total weight of the reduced catalyst, comprises at least 50% by weight of elements selected from rhenium, iron, ruthenium, cobalt, rhodium, iridium, nickel and copper.
24. A process for preparing hexamethylenediamine, in which
   a) muconic acid is subjected to a hydrogenation with hydrogen in the presence of an aqueous liquid A and in the presence of at least one transition metal catalyst C as defined in any of embodiments 9 to 12, to obtain adipic acid,
   b) the adipic acid obtained in step a) is subjected to a reaction with hydrogen in the presence of at least one hydrogenation catalyst to give hexane-1,6-diol,
   c) the hexane-1,6-diol obtained in step b) is subjected to an amination with ammonia in the presence of an amination catalyst to obtain hexamethylenediamine.
25. A process for preparing polyamide-6,6, in which
   a) muconic acid is subjected to a hydrogenation with hydrogen in the presence of an aqueous liquid A and in the presence of at least one transition metal catalyst C as defined in any of embodiments 9 to 12, to obtain adipic acid,
   b) the adipic acid obtained in step a) is subjected to a reaction with hydrogen in the presence of at least one hydrogenation catalyst to give hexane-1,6-diol,
   c) the hexane-1,6-diol obtained in step b) is subjected to an amination with ammonia in the presence of an amination catalyst to obtain hexamethylenediamine,
   d) the hexamethylenediamine obtained in step c) is subjected to a polycondensation with adipic acid to obtain polyamide-6,6.
26. The process according to embodiment 25, wherein the adipic acid used in step d) is prepared at least partly by the process according to any of embodiments 1 to 21.

### DESCRIPTION OF THE INVENTION

The process according to the invention is notable for the following advantages:
Muconic acid prepared from renewable raw materials is generally obtained in aqueous solutions. In the process according to the invention, hydrogenation is possible without a solvent exchange.
A preferred workup process on the industrial scale is recrystallization, and so no solvent exchange is required here either.
The aqueous adipic acid-containing mother liquors obtained in the workup can be recycled into the hydrogenation.

Adipic Acid

In the process according to the invention, hydrogenation is preferably effected using a muconic acid starting material consisting essentially of muconic acid. The muconic acid starting material used in the process according to the invention more preferably comprises at least 90% by weight, most preferably at least 95% by weight, based in each case on the total weight of the muconic acid starting material, of muconic acid.

The muconic acid usable in the process according to the invention may originate from renewable sources, which means natural sources such as sugars, e.g. starch, cellulose and glucose, or lignin. The preparation, for example, of muconic acid from, for example, starch, cellulose, glucose or lignin can be effected in all ways known to those skilled in the art, for example by biocatalytic means. The biocatalytic preparation of cis,cis-muconic acid by fermentation process is described for glucose, for example, in the prior art cited in the introductory part. The muconic acid usable in the process according to the invention may also originate from non-renewable sources. All muconic acids are suitable in principle for the process according to the invention, irrespective of the renewable or non-renewable source from which they originate and the synthesis route by which they have been prepared. Preferably, the muconic acid usable in accordance with the invention originates from renewable sources. Compounds obtained from renewable sources, for example muconic acid, have a different ¹⁴C-to-¹²C isotope ratio than compounds obtained from fossil sources such as mineral oil. The muconic acid which has been obtained from renewable sources and is used with preference accordingly preferably has a ¹⁴C-to-¹²C isotope ratio in the range from 0.5×10⁻¹² to 5×10⁻¹².

The term “muconic acid” in the context of the invention encompasses the different isomers of muconic acid, namely cis,cis-muconic acid, cis,trans-muconic acid and trans,trans-muconic acid, in any composition. Suitable feedstocks for the process according to the invention are all isomers of muconic acid. Preferably, the muconic acid used in the process according to the invention consists to an extent of at least 80% by weight, more preferably at least 90% by weight, of cis,cis-muconic acid, based on the total weight of all the muconic acid isomers present in the muconic acid used.

In the context of the invention, the term “muconic acid” refers to a starting material consisting essentially of fully protonated, underivatized muconic acid. Preferably, the muconic acid used for hydrogenation consists to an extent of at least 80% by weight, preferably to an extent of at least 95% by weight, especially to an extent of at least 99% by weight, of fully protonated, underivatized muconic acid.

The inventive hydrogenation of muconic acid may result in intermediates and by-products. Intermediates are the partly hydrogenated dihydromuconic acids still amenable to hydrogenation. By-products may result, for example, from addition of water onto one or both double bonds of the muconic acid used and any subsequent lactone formation. The adipic acid obtained by the process according to the invention may thus comprise at least one intermediate or by-product selected from the isomers of dihydromuconic acid, especially 2-hexenedicarboxylic acid and 3-hexenedicarboxylic acid, the saturated and unsaturated mono- and dilactones (III), (IV) and (V) of muconic acid, and mixtures comprising a plurality of these intermediates or by-products.

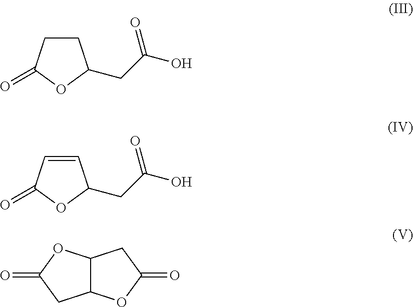

Preferably, the hydrogenation product obtained by the process according to the invention comprises not more than 5% by weight, more preferably not more than 2% by weight, of lactones of the formulae III to V, based on the total weight of the hydrogenation product.

The aqueous liquid A is a substance or substance mixture which forms a liquid phase under the hydrogenation conditions. The liquid A is essentially inert under the hydrogenation conditions, meaning that it is essentially not hydrogenated. Accordingly, the aqueous liquid A preferably does not have any ethylenically unsaturated double bonds.

The liquid A is preferably selected such that the adipic acid process product which forms dissolves in the liquid A under the hydrogenation conditions in a proportion of at least 1% by weight, preferably at least 10% by weight, more preferably at least 20% by weight, based on the total weight of the liquid A.

According to the invention, the liquid A comprises water or the liquid A consists of water. Preferably, the liquid A has a water content in the range from 1 to 100% by weight, more preferably 10 to 100% by weight, especially 50 to 100% by weight, based in each case on the total weight of the liquid A. Most preferably, the liquid A consists exclusively of water; accordingly, the liquid A most preferably has a water content in the range from 95 to 100% by weight, based on the total weight of the liquid A.

The aqueous liquid A is more preferably exclusively water. However, the aqueous liquid A may also comprise at least one organic solvent which is in liquid form under the hydrogenation conditions. Preferably, the organic solvents are at least partly miscible with water. Preferably, the organic solvent is selected from C₁-C₆-carboxylic acids, linear and cyclic, aliphatic and aromatic ethers, and mixtures thereof. Examples of preferred C₁-C₆-carboxylic acids are formic acid, acetic acid, propionic acid, butyric acid, valeric acid and mixtures thereof. Examples of preferred ethereal solvents are mono- and di-C₂-C₄-alkylene glycol C₁-C₄-alkyl ethers and C₄-C₈-cycloalkyl ethers, e.g. unsubstituted or C₁-C₄-alkyl-substituted tetrahydrofuran. Particularly preferred ethers are ethylene glycol dimethyl ether, ethylene glycol diethyl ether, diethylene glycol dimethyl ether (diglyme), diethylene glycol diethyl ether, propane-1,3-diol dimethyl ether, tetrahydrofuran, 2-methyltetrahydrofuran, 3-methyltetrahydrofuran, diphenyl ether, dioxane and mixtures thereof.

If the aqueous liquid A comprises at least one organic solvent, the proportion of the organic solvent is preferably 1 to 99% by weight, preferably 1 to 90% by weight, more preferably 1 to 50% by weight, based on the total weight of the liquid A. In a preferred embodiment of the process according to the invention, the proportion of organic solvent in the liquid A is less than 10% by weight, more preferably less than 5% by weight, especially less than 1% by weight, based on the total weight of the liquid A.

The muconic acid is at least partly insoluble in the liquid A under the hydrogenation conditions. Preferably, the mixture of muconic acid and liquid A is a suspension of muconic acid in the liquid A.

Preferably, the reaction mixture, at a minimum content of 50% by weight of water, based on the total weight of the reaction mixture, has a pH at 60° C. in the range from 1 to 6, preferably 1 to 5, more preferably 1 to 4.

Preferably, the liquid A has a water content in the range from 5 to 100% by weight, preferably 30 to 100% by weight, more preferably 50 to 100% by weight, particularly 65 to 10)% by weight, especially 95 to 100% by weight, based on the total weight of the liquid A.

Preferably, the muconic acid under the hydrogenation conditions has a solubility in the liquid A of preferably not more than 80 g/L, more preferably not more than 50 g/L.

Preference is given to using a suspension of the muconic acid in which the solids content of the muconic acid is at least 1% by weight, preferably at least 10% by weight, more preferably at least 20% by weight, based on the total weight of the liquid A and muconic acid. The solubility of muconic acid under the hydrogenation conditions in the liquid A can be ascertained by the person skilled in the art from literature values and optionally by simple experiments.

Preferably, the adipic acid obtained in the hydrogenation of muconic acid is discharged from the reaction zone together with the liquid A. For this purpose, it is advantageous when the adipic acid has a certain solubility in the liquid A. Preferably, in the process according to the invention, a liquid A in which adipic acid has a solubility under the reaction conditions of at least 50 g/L, preferably at least 100 g/L, is used.

The transition metal catalysts C used in the process according to the invention may in principle be any transition metal catalysts known to those skilled in the art for hydrogenation of carbon-carbon double bonds. In general, the transition metal catalyst C comprises at least one transition metal of groups 7, 8, 9, 10 and 11 of the IUPAC Periodic Table. Preferably, the transition metal catalyst C includes at least one transition metal from the group of Mn, Re, Fe, Ru, Co, Rh, Ir, Ni, Pd, Pt, Cu and Au. More preferably, the transition metal catalyst C includes at least one transition metal from the group of Re, Ru, Co, Rh, Ir and Ni. Most preferably, the transition metal catalyst C includes at least one transition metal from the group of Ni, Co and Rh. The transition metal catalysts C comprise said transition metals, especially the transition metals mentioned as preferred, generally as such, applied to a support, as precipitation catalysts, as Raney catalysts or as mixtures thereof. Specifically, the transition metal catalyst C is selected from Raney nickel, Raney cobalt, rhodium on a support material, for example rhodium on carbon, and mixtures thereof.

Inert support materials used for the inventive transition metal catalysts C may be virtually all prior art support materials as used advantageously in the production of supported catalysts, for example carbon, SiO₂ (quartz), porcelain, magnesium oxide, tin dioxide, silicon carbide, TiO₂ (rutile, anatase), Al₂O₃ (alumina), aluminum silicate, steatite (magnesium silicate), zirconium silicate, cerium silicate or mixtures of these support materials. Preferred support materials are carbon, aluminum oxide and silicon dioxide. A particularly preferred support material is carbon. The silicon dioxide support materials used for catalyst production may be silicon dioxide materials of different origin and production, for example fumed silicas or silicas produced by wet-chemical means, such as silica gels, aerogels or precipitated silicas (for production of the various SiO₂ starting materials see: W. Büchner, R. Schliebs, G. Winter, K. H. Büchel: IndustrieIle Anorganische Chemie [Industrial Inorganic Chemistry], 2nd ed., p. 532-533, VCH Verlagsgesellschaft, Weinheim 1986).

The transition metal catalysts C can be used in the form of shaped bodies, for example in the form of spheres, rings, cylinders, cubes, cuboids or other geometric bodies. Unsupported catalysts can be shaped by customary methods, for example by extrusion, tableting etc. The shape of supported catalysts is determined by the shape of the support. Alternatively, the support can be subjected to a shaping process before or after the application of the catalytically active component(s). The transition metal catalysts C can be used, for example, in the form of pressed cylinders, tablets, pellets, wagonwheels, rings, stars, or extrudates such as solid extrudates, polylobal extrudates, hollow extrudates and honeycombs, or other geometric bodies.

The catalyst particles generally have a mean (greatest) diameter of 0.5 to 20 mm, preferably 1 to 10 mm. These include, for example, transition metal catalysts C in the form of tablets, for example having a diameter of 1 to 7 mm, preferably 2 to 6 mm, and a height of 3 to 5 mm, in the form of rings having external diameter, for example, 4 to 7 mm, preferably 5 to 7 mm, height 2 to 5 mm and hole diameter 2 to 3 mm, or in the form of extrudates of different length having a diameter of, for example, 1.0 to 5 mm. Shapes of this kind can be obtained in a manner known per se by tableting or extrusion. For this purpose, it is possible to add customary auxiliaries to the catalyst composition, for example lubricants such as graphite, polyethylene oxide, cellulose or fatty acids (such as stearic acid) and/or shaping auxiliaries and reinforcing agents, such as fibers of glass, asbestos or silicon carbide.

The transition metal catalyst C may be in the form either of a homogeneous or heterogeneous catalyst under the hydrogenation conditions. Preferably, the transition metal catalyst C is in the form of a heterogeneous catalyst under the hydrogenation conditions. If a heterogeneous transition metal catalyst C is used, it may be applied, for example, to a support in mesh form. Alternatively or additionally, the heterogeneous transition metal catalyst C may also be applied to the inner wall of a tubular support, in which case the reaction mixture flows through the tubular support. Since the muconic acid is essentially in solid form in the liquid A, preference is given to configurations of the transition metal catalysts C and/or of the support to which the transition metal catalyst C has been applied which are not blocked and/or damaged by the particles of the muconic acid. Alternatively or additionally, the transition metal catalyst C can be used in the form of a particulate solid. In a preferred embodiment, the transition metal catalyst C is in the form of a suspension in the liquid A. If a liquid reaction output is removed from the reaction zone, the suspended transition metal catalyst C can be kept in the reaction zone by retention methods known to those skilled in the art. These retention methods preferably include crossflow filtration, gravitational filtration and/or filtration by means of at least one filter cartridge, for example in the form of a sintered metal frit.

The hydrogenation is preferably effected at a temperature in the range from 20° C. to 250° C., more preferably at a temperature in the range from 30° C. to 200° C., most preferably in the range from 40° C. to 150° C.

The inventive hydrogenation is preferably conducted at an absolute hydrogen pressure in the range from 1 to 300 bar, more preferably in the range from 1.5 to 200 bar, most preferably in the range from 2 to 100 bar.

The hydrogen used for hydrogenation may comprise one or more inert diluent gases, for example nitrogen and/or argon. Preferably, the hydrogen used for hydrogenation is used essentially in pure form, i.e. the hydrogen used for hydrogenation comprises generally less than 10% by weight, preferably less than 5% by weight, based on the total weight of the gas used for hydrogenation, of gases other than hydrogen.

The mean residence time of the reaction mixture in the reaction zone is generally in the range from 0.1 hour to 48 hours, preferably in the range from 0.2 hour to 24 hours, more preferably in the range from 0.3 hour to 10 hours.

According to the production volume, the process according to the invention can viably be conducted as a batchwise process, semi-batchwise process or continuous process. In the production of industrial volumes (>100 t), the continuous execution of the process according to the invention is preferred,

If the process according to the invention is conducted as a batchwise process, the procedure is generally to initially charge a reaction vessel with muconic acid, liquid A and transition metal catalyst C, and to inject hydrogen once. If the process according to the invention is conducted as a semi-batchwise process, the procedure will generally be to initially charge a reaction vessel with muconic acid, liquid A and transition metal catalyst C, and to feed in hydrogen continuously. On completion of reaction, the adipic acid solution obtained will generally be discharged from the reaction vessel and optionally subjected to a workup, preferably the same workup as the portion of the reaction mixture withdrawn from the reaction zone in the continuous process. The transition metal catalyst C can optionally be removed by the retaining devices and/or retaining methods mentioned and preferably used in at least one further inventive batchwise or semi-batchwise process.

The process according to the invention is preferably conducted as a continuous process. In this case, reaction zone is continuously supplied with muconic acid, liquid A, hydrogen and optionally transition metal catalysts C, and at least a portion of the adipic acid-containing reaction mixture is withdrawn continuously. The muconic acid is fed to the reaction zone as a solid or as a suspension, preferably without addition of solvent. It will be appreciated that the supply of muconic acid, if it is to be introduced into the reaction zone as a solid without solvent, is effected at a separate place and/or time from the supply of liquid A into the reaction zone. If the muconic acid is to be introduced into the reaction zone as a suspension, the reaction zone preferably has at least one upstream vessel in which, for example, the suspension of the muconic acid, preferably in the liquid A, is prepared with stirring or pumped circulation. If the transition metal catalyst C is used in suspended form, the production of the transition metal catalyst suspension can be conducted together with the production of the suspension of muconic acid in the at least one upstream reaction vessel.

In a preferred embodiment of the process according to the invention, at least a portion of the reaction mixture is withdrawn from the reaction zone and the reaction mixture withdrawn is subjected to a separation into an adipic acid-enriched fraction and an adipic acid-depleted fraction. Suitable means for separation into an adipic acid-enriched fraction and an adipic acid-depleted fraction are the separation methods known in principle to those skilled in the art, preferably selected from crystallization methods, distillation methods, adsorption methods, ion exchange methods, membrane separation methods, extraction methods or a combination of two or more of these methods. More preferably, the separation into an adipic acid-enriched fraction and an adipic acid-depleted fraction comprises a one-stage or multistage process for at least partial crystallization of the adipic acid. The crystallization is preferably conducted at temperatures from 10 to 80° C.

Preferably, the adipic acid-depleted fraction is at least partly recycled into the reaction zone. In a preferred configuration, the reaction mixture withdrawn from the reaction zone is at least partly subjected to a crystallization of the adipic acid and at least a portion of the adipic acid-depleted supernatant (of the mother liquor) is returned to the reaction zone. The crystallization of the adipic acid can also be effected in two or more stages. In order to prevent accumulation of impurities, a portion of the mother liquor can be discharged.

In a further configuration, a homogeneous transition metal catalyst C, i.e. one essentially dissolved in the liquid, is used, which is at least partly recycled into the reaction zone together with the mother liquor. In a further configuration of this embodiment, the homogeneous transition metal catalyst C is recovered from the portion of the reaction mixture withdrawn from the reaction zone by extraction methods known in principle to the person skilled in the art. The portion of the homogeneous transition metal catalyst C recovered can optionally be recycled into the reaction zone.

Preferably, the muconic acid used originates from renewable sources. According to the purity of the muconic acid used, it may also comprise substances which act as a catalyst poison to the transition metal catalyst C. These may be compounds comprising sulfur, phosphorus, nitrogen and/or halogens. There may therefore be a need to replace the spent transition metal catalyst C continuously with unused, still-reactive transition metal catalyst C. If the transition metal catalyst C is to be replaced continuously with unused, still-reactive transition metal catalyst C, preference is given in the process according to the invention to transition metal catalysts C present as a suspension in the liquid A. If a suspended transition metal catalyst C is used and the process is conducted as a continuous process, particular preference is accordingly given to embodiments of the process according to the invention in which at least a portion of the suspended transition metal catalyst C is continuously removed from the reaction zone, for example by filtration methods or partial or full depletion from a portion of the reaction mixture withdrawn from the reaction zone, with or without recycling of the portion of the reaction mixture withdrawn and depleted of transition metal catalyst C, and in which still-reactive transition metal catalyst C is supplied continuously to the reaction zone. The continuous supply of transition metal catalyst C to the reaction zone can be effected in the form of a solid or in the form of a suspension, preferably of a suspension in the liquid A.

The catalyst hourly space velocity in continuous mode is preferably 0.01 to 100 kg, more preferably 0.1 to 50 kg, of muconic acid to be hydrogenated per kg of transition metal catalyst C and hour.

The molar ratio of hydrogen to muconic acid compound is preferably 2:1 to 20:1, more preferably 2:1 to 3:1.

In a specific execution of the process according to the invention, the hydrogenation is effected in n series-connected hydrogenation reactors, where n is an integer of at least 2. Suitable values of n are 2, 3, 4, 5, 6, 7, 8, 9 and 10. Preferably, n is 2 to 6 and especially 2 or 3. In this execution, the hydrogenation is preferably effected continuously.

The reactors used for hydrogenation may each independently have one or more reaction zones within the reactor. The reactors may be identical or different reactors. These may, for example, each have the same or different mixing characteristics and/or be divided once or more than once by internals.

Suitable pressure-resistant reactors for the hydrogenation are known to those skilled in the art. These include the reactors generally customary for gas-liquid reactions, for example tubular reactors, shell and tube reactors, gas circulation reactors, bubble columns, loop apparatuses, stirred tanks (which may also be configured as stirred tank cascades), airlift reactors etc.

The process according to the invention using heterogeneous transition metal catalysts C can be conducted in fixed bed mode or suspension mode. Operation in fixed bed mode can be conducted, for example, in liquid phase mode or in trickle mode. In this case, the transition metal catalysts C are preferably used in the form of shaped bodies as described above, for example in the form of pressed cylinders, tablets, pellets, wagonwheels, rings, stars, or extrudates such as solid extrudates, polylobal extrudates, hollow extrudates, honeycombs etc.

Fixed bed reactors are unsuitable for the hydrogenation of solids-containing mixtures, for example of muconic acid suspensions. However, they can be used in the process according to the invention, for example, as postreactors into which a homogeneous liquid phase is fed.

In suspension mode, heterogeneous catalysts are likewise used. The heterogeneous catalysts are usually used in a finely divided state and are in fine suspension in the reaction medium.

Suitable heterogeneous catalysts and processes for preparation thereof have been described above.

In the case of hydrogenation over a fixed bed, a reactor with a fixed bed arranged in the interior thereof, through which the reaction medium flows, is used. This fixed bed may be formed from a single bed or from a plurality of beds. Each bed may have one or more zones, at least one of the zones comprising a material active as a hydrogenation catalyst. Each zone may have one or more different catalytically active materials and/or one or more different inert materials. Different zones may each have identical or different compositions. It is also possible to provide a plurality of catalytically active zones separated from one another, for example, by inert beds. The individual zones may also have different catalytic activity. To this end, it is possible to use different catalytically active materials and/or to add an inert material to at least one of the zones. According to the invention, the reaction medium which flows through the fixed bed comprises at least one liquid phase, namely the inert liquid A. The reaction medium may also additionally comprise a gaseous phase.

The reactors used in the hydrogenation in suspension are especially loop apparatuses such as jet loops or propeller loops, stirred tanks, which may also be configured as stirred tank cascades, bubble columns or airlift reactors.

If desired, in a hydrogenation apparatus composed of n reactors, at least two of the reactors (i.e. 2 to n of the reactors) may have different temperatures. In a specific embodiment, every downstream reactor is operated with a higher temperature than the previous reactor. In addition, each of the reactors may have two or more reaction zones with different temperatures. For example, a different temperature, preferably a higher temperature, can be established in a second reaction zone than in the first reaction zone, or a higher temperature than in an upstream reaction zone can be established in every downstream reaction zone, for example in order to achieve substantially full conversion in the hydrogenation.

If desired, in a hydrogenation apparatus composed of n reactors, at least two of the reactors (i.e. 2 to n of the reactors) may have different pressures. In a specific embodiment, every downstream reactor is operated with a higher pressure than the previous reactor.

The hydrogen required for the hydrogenation can be fed into the first and optionally additionally into at least one further reactor. Preferably, hydrogen is fed only into the first reactor. The amount of hydrogen fed to the reactors is calculated from the amount of hydrogen consumed in the hydrogenation reaction and any amount of hydrogen discharged with the offgas.

The proportion of muconic acid which has been converted in the particular reactor can be adjusted, for example, via the reactor volume and/or the residence time in the reactor. The conversion in the first reactor, based on muconic acid, is preferably at least 60%, more preferably at least 70%. The overall conversion in the hydrogenation of the muconic acid, based on the muconic acid, is preferably at least 97%, more preferably at least 98%, especially at least 99%.

To remove the heat of reaction which arises in the exothermic hydrogenation, it is possible to provide one or more of the reactors with at least one cooling apparatus. In a specific embodiment, at least the first reactor is provided with a cooling apparatus. The heat of reaction can be removed by cooling of an external circulation stream or by internal cooling in at least one of the reactors. For the internal cooling, it is possible to use the apparatus customary for this purpose, generally hollow modules such as Field tubes, tube coils, heat exchanger plates, etc. Alternatively, the reaction can also be effected in a cooled shell and tube reactor.

Preferably, the hydrogenation is effected in n series-connected hydrogenation reactors, where n is an integer of at least two, and wherein at least one reactor has a stream from the reaction zone conducted within an external circuit (external circulation stream, liquid circulation system, loop mode). Preferably, n is two or three.

Preferably, the hydrogenation is effected in n series-connected hydrogenation reactors, where n is preferably two or three, and the 1st to (n-1)th reactor has a stream from the reaction zone conducted within an external circuit.

Preferably, the hydrogenation is effected in n series-connected hydrogenation reactors, where n is preferably two or three, and wherein the reaction is conducted adiabatically in the nth reactor (the last reactor through which the reaction mixture to be hydrogenated flows).

Preferably, the hydrogenation is effected in n series-connected hydrogenation reactors, where n is preferably two or three, and wherein the nth reactor is operated in straight pass.

If a reactor is operated “in straight pass”, this shall be understood here and hereinafter to mean that a reactor is operated without recycling of the reaction product in the manner of a loop mode of operation. The mode of operation in straight pass does not fundamentally rule out backmixing internals and/or stirring units in the reactor.

When the reaction mixture hydrogenated in one of the reactors connected downstream of the first reactor (i.e. in the 2nd to nth reactor) has only such low proportions of hydrogenatable muconic acid or intermediates that the exothermicity occurring in the reaction is insufficient to maintain the desired temperature in the reactor, heating of the reactor (or of individual reaction zones of the second reactor) may also be required. This can be effected analogously to the above-described removal of the heat of reaction by heating an external circulation stream or by internal heating. In a suitable embodiment, the temperature of a reactor can be controlled by using the heat of reaction from at least one of the upstream reactors.

In addition, the heat of reaction withdrawn from the reaction mixture can be used to heat the feed streams to the reactors. For this purpose, for example, the feed stream of the muconic acid into the first reactor can be mixed at least partly with an external circulation stream of this reactor and then the combined streams can be conducted into the first reactor. In addition, in the case of m=2 to n reactors, the feed stream from the (m-1)th reactor can be mixed in the mth reactor with a circulation stream of the mth reactor, and the combined streams can then be conducted into the mth reactor. In addition, the feed stream of the muconic acid and/or another feed stream can be heated with the aid of a heat exchanger which is operated with heat of hydrogenation withdrawn.

In a specific configuration of the process, a reactor cascade composed of n series-connected reactors is used, in which case the reaction is performed adiabatically in the nth reactor. In the context of the present invention, this term is used in the technical and not in the physicochemical sense. Thus, the reaction mixture generally experiences a temperature increase as it flows through the second reactor owing to the exothermic hydrogenation reaction. An adiabatic reaction regime is understood to mean a procedure in which the amount of heat released in the hydrogenation is absorbed by the reaction mixture in the reactor and no cooling by cooling apparatuses is employed. The heat of reaction is thus removed from the second reactor with the reaction mixture, apart from a residual fraction which is released to the environment by natural heat conduction and heat emission from the reactor. The nth reactor is preferably operated in straight pass.

In a preferred embodiment, the hydrogenation is effected using a two-stage reactor cascade, in which case the first hydrogenation reactor has a stream from the reaction zone conducted within an external circuit. In a specific embodiment of the process, a reactor cascade composed of two series-connected reactors is used, in which case the reaction is performed adiabatically in the third reactor.

In a further preferred embodiment, the hydrogenation is effected using a three-stage reactor cascade, in which case the first and second hydrogenation reactors have a stream from the reaction zone conducted within an external circuit. In a specific embodiment of the process, a reactor cascade composed of three series-connected reactors is used, in which case the reaction is performed adiabatically in the third reactor. An adiabatic reaction regime is understood to mean a procedure in which the amount of heat released in the hydrogenation is absorbed by the reaction mixture in the reactor and no cooling by cooling apparatuses is employed.

In one embodiment, additional mixing can be effected in at least one of the reactors used. Additional mixing is especially advantageous when the hydrogenation is effected with long residence times of the reaction mixture. Mixing can be effected, for example, using the streams introduced into the reactors, by introducing them into the particular reactors using suitable mixing devices, such as nozzles. Mixing can also be effected using streams from the particular reactor conducted within an external circuit.

To complete the hydrogenation, an output which still comprises hydrogenatable muconic acid and/or intermediates is withdrawn from each of the first to (n-1)th reactors and is fed into the downstream hydrogenation reactor in each case. In a specific embodiment, the output is separated into a first and a second substream, in which case the first substream is fed back as a circulation stream to the reactor from which it has been withdrawn, and the second substream is fed to the downstream reactor. The output may comprise dissolved or gaseous fractions of hydrogen. In a specific embodiment, the output from the first to (n-1)th reactor is fed to a phase separation vessel and separated into a liquid phase and a gaseous phase, the liquid phase is separated into the first and the second substream, and the gas phase is fed separately at least partly to the downstream reactor. In an alternative embodiment, the output from the first to (n-1)th reactor is fed to a phase separation vessel and separated into a first liquid hydrogen-depleted substream and a second hydrogen-enriched substream. The first substream is then fed back as a circulation stream to the reactor from which it has been withdrawn, and the second substream is fed to the downstream reactor (as feed containing muconic acid compound M and hydrogen). In a further alternative embodiment, the second to nth reactor is charged with hydrogen not via a hydrogenous feed withdrawn from the upstream reactor but rather with fresh hydrogen via a separate feed line.

The above-described process variant is particularly advantageously suitable for control of the reaction temperature and of the heat transfer between reaction medium, delimiting apparatus walls and environment. A further means of controlling the heat balance consists in regulating the entry temperature of the muconic acid. For instance, a lower temperature of the incoming feed generally leads to improved removal of the heat of hydrogenation. When the catalyst activity declines, the entry temperature can be selected at a higher level in order to achieve a higher reaction rate and thus to compensate for the decline in catalyst activity. Advantageously, it is generally possible in this way to prolong the service life of the transition metal catalyst C used.

Hexane-1,6-diol

The invention also relates to a process for preparing hexane-1,6-diol, in which
a) muconic acid is subjected to a hydrogenation with hydrogen in the presence of an aqueous liquid A and in the presence of at least one transition metal catalyst C as defined above to obtain adipic acid,
b) the adipic acid obtained in step a) is subjected to a hydrogenation in the presence of a hydrogenation catalyst to obtain a hexane-1,6-diol.

The process according to the invention is especially suitable for preparation of hexane-1,6-diol from natural raw material sources. A hexane-1,6-diol prepared completely from natural raw material sources generally has a ¹⁴C-to-¹²C isotope ratio in the range from 0.5×10⁻¹² to 5×10⁻¹².

The hydrogenation of adipic acid to hexane-1,6-diol is known in principle. It is preferably effected in the liquid phase. This hydrogenation can be effected without the addition of an external solvent or in the presence of an external solvent. Suitable external solvents are preferably selected from water, aliphatic C₁-C₅ alcohols (especially selected from methanol, ethanol, n-propanol, i-propanol, n-butanol, sec-butanol, i-butanol and tert-butanol), aliphatic C₂-C₆ α,ω-diols (i.e. ethylene glycol, propane-1,3-diol, butane-1,4-diol, pentane-1,5-diol or hexane-1,6-diol), ethers (especially selected from tetrahydrofuran, 2-methyltetrahydrofuran, diethyl ether and methyl tert-butyl ether) and mixtures thereof. Preference is given to aliphatic C₁-C₅ alcohols, water and mixtures of these solvents. Particular preference is given to methanol, n-butanol, isobutanol, water and mixtures of these solvents. It is additionally preferable to use the hexane-1,6-diol target product as the solvent. In this case, hexane-1,6-diol can be used alone or in a mixture with alcohols and/or water.

It is preferable that, for the catalytic hydrogenation in step b), a solution comprising 10 to 60% by weight, more preferably 20 to 50% by weight, most preferably 30 to 50% by weight, of adipic acid is used.

Preferably, the hydrogenation catalyst used in step b), based on the total weight of the reduced catalyst, comprises at least 50% by weight of elements selected from rhenium, iron, ruthenium, cobalt, rhodium, iridium, nickel and copper.

For the inventive hydrogenation in step b), preference is given to using catalysts comprising at least 50% by weight of cobalt and at least 0.1% by weight of ruthenium and/or at least 0.1% by weight of rhenium, based on the total weight of the reduced catalyst. Catalysts comprising at least 50% by weight of cobalt may further comprise especially phosphoric acid and/or further transition metals such as copper, manganese and/or molybdenum.

The preparation of a suitable cocatalyst precursor is known from DE 2 321 101. This comprises, in the unreduced, calcined state, 40 to 60% by weight of cobalt (calculated as Co), 13 to 17% by weight of copper (calculated as Cu), 3 to 8% by weight of manganese (calculated as Mn), 0.1 to 5% by weight of phosphates (calculated as H₃PO₄) and 0.5 to 5% by weight of molybdenum (calculated as MoO₃). EP 636 409 B1 describes the preparation of further suitable cobalt catalyst precursors consisting to an extent of 55 to 98% by weight of cobalt, to an extent of 0.2 to 15% by weight of phosphorus, to an extent of 0.2 to 15% by weight of manganese and to an extent of 0.2 to 15% by weight of alkali metals (calculated as oxide). Catalyst precursors of this kind can be reduced to the active catalysts comprising metallic cobalt by treatment with hydrogen or mixtures of hydrogen and the inert gases such as nitrogen. These catalysts are unsupported catalysts consisting very predominantly of metal and not comprising any catalyst support.

Useful catalysts are in principle all homogeneous and heterogeneous catalysts suitable for hydrogenation of carbonyl groups, such as metals, metal oxides, metal compounds or mixtures thereof. Examples of homogeneous catalysts are described, for example, in Houben-Weyl, Methoden der Organischen Chemie [Methods of Organic Chemistry], volume IV/1c, Georg Thieme Verlag Stuttgart, 1980, p. 45-67, and examples of heterogeneous catalysts are described, for example, in Houben-Weyl, Methoden der Organischen Chemie, volume IV/1c, p. 16 to 26.

Preference is given to using catalysts comprising one or more elements from groups 3 and 6 to 11 of the Periodic Table of the Elements (IUPAC), preferably copper, chromium, molybdenum, manganese, rhenium, ruthenium, cobalt, nickel or palladium, more preferably copper, cobalt or rhenium.

The catalysts may consist solely of active components, or the active components thereof may be applied to supports. Suitable support materials are especially Cr₂O₃, Al₂O₃, SiO₂ and ZrO₂, or mixtures thereof.

Particular preference is given to catalysts as described in EP 0 552 463 A1. These are catalysts which, in the oxidic form, have the composition

CuₐAl_{b}Zr_{c}Mn_{d}Oₓ

where a>0, b>0, c≧0, d>0, a>b/2, b>a/4, a>c and a>d, and x denotes the proportion of oxygen ions required per formula unit to give electronic neutrality. These catalysts can be prepared, for example, according to the specifications of EP 552 463 A1, by precipitation of sparingly soluble compounds from solutions comprising the corresponding metal ions in the form of salts thereof. Suitable salts are, for example, halides, sulfates and nitrates. Suitable precipitants are all agents which lead to the formation of those insoluble intermediates that can be converted to the oxides by thermal treatment. Particularly suitable intermediates are hydroxides and carbonates or hydrogencarbonates, and so alkali metal carbonates or ammonium carbonate are used as particularly preferred precipitants. Thermal treatment of the intermediates is effected at temperatures in the range from 500° C. to 1000° C. The BET surface area of such catalysts is between 10 and 150 m²/g.

Additionally suitable are catalysts which have a BET surface area of 50 to 120 m²/g, fully or partly comprise crystals having spinel structure, and comprise copper in the form of copper oxide.

WO 2004/085 356 A1 also describes copper catalysts suitable for the process according to the invention, which comprise copper oxide, aluminum oxide and at least one of the oxides of lanthanum, tungsten, molybdenum, titanium or zirconium, and additionally pulverulent metallic copper, copper flakes, pulverulent cement, graphite or a mixture thereof.

The hydrogenation of the adipic acid to hexane-1,6-diol in step b) is effected preferably at a temperature in the range from 160 to 240° C., more preferably 170 to 230° C., most preferably 170 to 220° C.

In a preferred embodiment of the process according to the invention for preparing hexane-1,6-diol from muconic acid, the hydrogenation of muconic acid is effected in a first loop reactor in step a), and the hydrogenation of the adipic acid obtained in step a) in a second loop reactor in step b). Preferably, the hydrogenation product obtained in step b) is post-hydrogenated in a downstream tubular reactor which is operated in straight pass.

A loop reactor is understood to mean a reactor in which the reactor contents are circulated. After flowing through the reactor, the feed can be cooled in a cooling apparatus, for example a heat exchanger, a substream of the cooled stream can be recycled into the reactor, and the residual stream can be passed into the next process stage. The circuit may be an internal or external circuit. Preferably, the external circulation stream can be cooled in a cooling apparatus, for example a heat exchanger. Preferred heat exchangers are plate heat exchangers, shell and tube heat exchangers or double tube heat exchangers. By removing the heat of reaction, the temperature rise in the reactor can be controlled efficiently in the course of the exothermic hydrogenation. The mean residence time in the loop reactor is preferably 0.1 to 10 h, more preferably 0.2 to 4 h.

In a further preferred embodiment of the process according to the invention, steps a) and b) take place in the same reactor, in which case two reaction zones at different temperatures are present in this loop reactor. Preferably, in this embodiment, there is a downstream tubular reactor operated in straight pass, in which the hydrogenation product obtained in step b) is post-hydrogenated.

Preferably, the hydrogenations in the loop reactors are effected in liquid phase mode or trickle mode.

The reaction output obtained in the hydrogenation of adipic acid in water as solvent is an aqueous hexane-1,6-diol solution. After the cooling and decompression of the hydrogenation output, the water is generally removed by distillation, and hexane-1,6-diol can be obtained in high purity (>97%).

The invention also relates to a process for preparing hexamethylenediamine, in which

Hexamethylenediamine
a) muconic acid is subjected to a hydrogenation with hydrogen in the presence of an aqueous liquid A and in the presence of at least one transition metal catalyst C as defined above and hereinafter, to obtain adipic acid,
b) the adipic acid obtained in step a) is subjected to a reaction with hydrogen in the presence of at least one hydrogenation catalyst to give hexane-1,6-diol,
c) the hexane-1,6-diol obtained in step b) is subjected to an amination with ammonia in the presence of an amination catalyst to obtain hexamethylenediamine.

With regard to process steps a) and b), reference is made to the above remarks regarding these steps in full.

In step c), the hexane-1,6-diol obtained in step b) is preferably reacted with ammonia in the presence of an amination catalyst to give hexamethylenediamine.

The hexamethylenediamine synthesized in the process according to the invention generally has a ¹⁴C-to-¹²C isotope ratio in the range from 0.5×10⁻¹² to 5×10⁻¹².

The inventive amination can be conducted without supply of hydrogen, but preferably with supply of hydrogen.

In one embodiment of the invention, the catalysts used are preferably predominantly cobalt, silver, nickel, copper or ruthenium, or mixtures of these metals. “Predominantly” is understood here to mean that one of these metals is present to an extent of more than 50% by weight in the catalyst (calculated without support). The catalysts can be used in the form of unsupported catalysts, i.e. without catalyst support, or in the form of supported catalysts. The supports used are preferably SiO₂, Al₂O₃, TiO₂, ZrO₂, activated carbon, silicates and/or zeolites. Said catalysts are preferably used in the form of fixed bed catalysts. It is also possible to use cobalt, nickel and/or copper in the form of suspension catalysts of the Raney type.

In one embodiment of the invention, the hexane-1,6-diol is aminated in homogeneous phase and the catalyst is a complex catalyst comprising at least one element selected from groups 8, 9 and 10 of the Periodic Table (IUPAC) and at least one donor ligand. Catalysts of this kind are known, for example, from WO 2012/119929 A1.

The amination is effected preferably at temperatures of 100 to 250° C., more preferably 120 to 230° C., most preferably 100 to 210° C.

The total pressure is preferably in the range from preferably 5 to 30 MPa, more preferably 7 to 27 MPa and most preferably 10 to 25 MPa.

The molar ratio of hexane-1,6-diol to ammonia is preferably 1:30, more preferably 1:25, most preferably 1:20.

The amination can be effected without solvent. However, it is preferably conducted in the presence of at least one solvent. Preferred solvents are water, ethers or mixtures of these solvents, and ether is more preferably selected from dioxane, tetrahydrofuran, 2-methyltetrahydrofuran, dioxolane, dibutyl ether and methyl tert-butyl ether.

In a preferred embodiment of the process according to the invention, the aqueous hexane-1,6-diol solutions obtained in the hydrogenation of muconic acid are used in the amination step without workup.

In a particularly preferred embodiment, the amination is conducted in the presence of hexamethyleneimine as a solvent or hexamethyleneimine/water mixtures.

The amount of solvent is preferably such as to give rise to 5 to 80%, preferably 10 to 70%, more preferably 15 to 60%, by weight hexane-1,6-diol solutions.

Preferably 10 to 150 liters, more preferably 10 to 100 liters, of hydrogen are supplied per mole of hexane-1,6-diol.

The partial hydrogen pressure is preferably in the range from preferably 1 to 40 MPa, more preferably 5 to 30 MPa and most preferably 10 to 25 MPa.

In one embodiment of the invention, the amination of hexane-1,6-diol with ammonia, in a first component step c1), is effected to give a mixture of 1-amino-6-hydroxyhexane and hexamethylenediamine, comprising more than 50% by weight of 1-amino-6-hydroxyhexane. In a component step c2), the latter is separated together with hexa-methylenediamine from unconverted hexane-1,6-diol and, in a component step c3), reacted with further ammonia to give hexamethylenediamine.

The amination can be conducted batchwise or continuously, in the liquid or gas phase, preference being given to a continuous process regime.

The workup of the hexamethylenediamine target product still comprising 1-amino-6-hydroxyhexane is preferably effected by distillation. Since 1-amino-6-hydroxyhexane and hexamethylenediamine have very similar vapor pressures, pure hexamethylene diamine is discharged. Mixtures of 1-amino-6-hydroxyhexane and hexamethylene diamine are recycled into the distillation stage.

Polyamide-6,6

The invention also relates to a process for preparing polyamide-6,6, in which
a) muconic acid is subjected to a hydrogenation with hydrogen in the presence of an aqueous liquid A and in the presence of at least one transition metal catalyst C as defined above and hereinafter, to obtain adipic acid,
b) the adipic acid obtained in step a) is subjected to a reaction with hydrogen in the presence of at least one hydrogenation catalyst to give hexane-1,6-diol,
c) the hexane-1,6-diol obtained in step b) is subjected to an amination with ammonia in the presence of an amination catalyst to obtain hexamethylene diamine
d) the hexamethylenediamine obtained in step c) is subjected to a polycondensation with adipic acid to obtain polyamide-6,6.

Processes for preparing polyamide-6,6 (nylon, polyhexamethyleneadipamide) are known in principle to those skilled in the art. Polyamide-6,6 is prepared predominantly by polycondensation of what are called AH salt solutions, i.e. of aqueous solutions comprising adipic acid and 1,6-diaminohexane (hexamethylenediamine) in stoichiometric amounts. Conventional preparation processes for polyamide-6,6 are described, for example, in Kunststoffhandbuch, 3/4 Technische Thermoplaste: Polyamide [Plastics Handbook, 3/4 Industrial Thermoplastics: Polyamides], Carl Hanser Verlag, 1998, Munich, p. 42-71. More particularly, it is also possible to work by a method known from Hans-Georg Elias, Makromoleküle [Macromolecules], 4th edition, pages 796 to 797, Hüthig-Verlag (1981). The aforementioned documents are hereby fully incorporated by reference.

The inventive preparation of polyamide-6,6 preferably comprises:
d1) reacting adipic acid and hexamethylenediamine in a molar ratio of about 1:1 to give hexamethylenediammonium adipate (AH salt), and
d2) converting the hexamethylenediammonium adipate to polyamide-6,6.

The conversion of the hexamethylenediammonium adipate to polyamide-6,6 in step d2) is effected especially in the presence of water at a temperature of not more than 280° C., more preferably of not more than 275° C.

The process according to the invention is especially suitable for partial or full preparation of polyamide-6,6 from natural raw material sources. An essential aspect of the present invention is thus the ecologically and economically improved provision of adipic acid, of hexamethylenediamine and of polyamide-6,6 prepared therefrom from natural muconic acid sources. A polyamide-6,6 prepared completely from natural raw material sources generally has a ¹⁴C-to-¹²C isotope ratio in the range from 0.5×10⁻¹² to 5×10⁻¹².

In a preferred embodiment for preparation of polyamide-6,6, a hexamethylenediamine prepared by the process according to the invention, comprising steps a) to c), is polycondensed with an adipic acid prepared by step a) of the process according to the invention to give polyamide-6,6.

The examples which follow serve to elucidate the invention and should not be understood in a limiting manner.

### EXAMPLES

Feedstocks Used:

cis,cis-muconic acid (from Aldrich)

water

hydrogen

Raney nickel

Raney cobalt

2% rhodium on carbon

### Experimental Method for Examples 1 to 3

TABLE 1

| | Transition | Adipic | Muconic | Lactones III | Bislactone |
|---|---|---|---|---|---|
| | metal | acid | acid (% by | and IV | V |
| | catalyst C | (% by wt.) | wt.) | (% by wt.) | (% by wt.) |
| Example 1 | Raney Ni | 95 | 0 | 5 | 0 |
| Example 2 | Raney Co | 95 | 0 | 5 | 0 |
| Example 3 | 2% Rh/C | 98 | 0 | 2 | 0 |

A suspension was prepared from 24 g of cis,cis-muconic acid, 56 g of water and 1 g of the transition metal catalyst C specified in table 1, and the suspension was introduced into a 300 stirred autoclave made from 1.4571 stainless steel. Hydrogen was injected to 30 bar, the stirrer was switched on (700 rpm) and the mixture was heated to 80° C. over a period of 20 min. Once the mixture had been heated to 80° C., the hydrogen pressure was increased to 100 bar and this hydrogen pressure was maintained by metering in further hydrogen over the reaction time. After a reaction time of 12 hours, the reaction mixture was cooled to about 60° C., the pressure was reduced to standard pressure, and the catalyst was filtered out of the reaction mixture. The filtrate was analyzed by means of ¹H NMR spectroscopy. By means of ¹H NMR spectroscopy, the yields of adipic acid, the amounts of unconverted cis,cis-muconic acid and the amounts of by-products formed (compounds of the formulae III, IV and V) were determined.

### Example 4

15 g of 2% Rh/C catalyst were suspended in 150 mL of water and the suspension was introduced into a 250 mL reactor. The suspension was stirred (700 rpm) and heated to 80° C. (internal reactor temperature). This temperature was kept constant over the reaction time by means of a temperature control device mounted in the reactor. 30 g/hour of a 33% by weight suspension of cis,cis-muconic acid in water and 50 standard liters/hour of hydrogen gas were conducted continuously into the reactor. At the same time, liquid and excess gas were conducted continuously out of the reactor, and the liquid volume and the pressure (about 50 bar) in the reactor were kept constant. Upstream of the discharge orifice, the reactor had a sintered metal frit (pore diameter 5 micrometres), which retained the suspended catalyst particles and muconic acid particles in the reactor. Beyond the discharge orifice was the discharge line. The discharge line had a valve which was used to decompress the mixture of discharged reaction mixture and discharged gas to standard pressure at 80° C. The pH of the reaction solution, measured at 60° C., was about 3. In total, about 5 kg of the cis,cis-muconic acid suspension were converted in the reactor. The liquid discharged was analyzed by means of ¹H NMR spectroscopy. According to ¹H NMR analysis, the discharged liquid, after removal of the water, comprised 96% by weight of adipic acid, 0.5% by weight of muconic acid, 2% by weight of dihydromuconic acid and 1% by weight of lactone I. Subsequently, the liquid discharged was purified by crystallization, or a post-hydrogenation was conducted with subsequent crystallization.

Crystallization:

The liquid discharged from the reactor was purified by crystallization. For this purpose, 1 kg of liquid discharged was cooled gradually from 60° C. to 20° C., in the course of which adipic acid crystallized out. The crystals were filtered off and dried. About 300 g of adipic acid were obtained with a purity of 99.85%. The adipic acid thus obtained was dissolved in 600 g of water, the solution was heated to 80° C. and the solution was cooled gradually to 20° C., in the course of which the adipic acid crystallized out. The crystals were filtered off and dried. About 270 g of adipic acid were obtained with a purity of 99.92%. The mother liquors were each recycled into the hydrogenation.

Post-Hydrogenation and Crystallization:

The liquid discharged from the reactor was post-hydrogenated and purified by crystallization. 4 kg of the liquid discharged were post-hydrogenated in a trickle bed reactor at hydrogen pressure 50 bar over 200 mg of 2% Rh/C at 150° C. In the course of this, the trickle bed reactor was supplied with 50 g/hour of the liquid discharged and 20 standard liters of hydrogen gas/hour. In the reaction output from the trickle bed, it was no longer possible to detect any ethylenically unsaturated compounds by ¹H NMR analysis. The liquid discharged from the trickle bed reactor was cooled to 80° C. and the liquid was cooled gradually to 20° C., in the course of which adipic acid crystallized out. The crystals were filtered off and dried. About 310 g of adipic acid were obtained with a purity of 99.95%.

1.-23. (canceled)

24. A process for preparing adipic acid or at least one conversion product thereof, selected from hexane-1,6-diol, hexamethylenediamine and polyamide-6,6, in which muconic acid is hydrogenated with hydrogen in the presence of at least one heterogeneous transition metal catalyst C and of an aqueous liquid A in a reaction zone, wherein the liquid A has a water content in the range from 65 to 100% by weight, based on the total weight of the liquid A and wherein the muconic acid is at least partly insoluble in the liquid A under the hydrogenation conditions and wherein the transition metal catalyst C includes at least one transition metal selected from the group consisting of Ru, Co, Rh, Ir, Ni, Fe, Pd, Pt, Cu and Au.

25. The process according to claim 24, wherein the reaction mixture, at a minimum content of 50% by weight of water, based on the total weight of the reaction mixture, has a pH at 60° C. in the range from 1 to 6.

26. The process according to claim 24, wherein the liquid A has a water content in the range from 95 to 100% by weight, based on the total weight of the liquid A.

27. The process according to claim 24, wherein the hydrogenation is conducted continuously.

28. The process according claim 24, wherein the muconic acid under the hydrogenation conditions has a solubility in the liquid A of not more than 50 g/L.

29. The process according to claim 24, wherein the muconic acid is partly in the form of particles suspended in the liquid A in the hydrogenation.

30. The process according to claim 24, wherein hydrogenation is effected using a liquid A in which adipic acid has a solubility under the reaction conditions of at least 100 g/L.

31. The process according to claim 24, wherein the muconic acid originates from renewable sources.

32. The process according to claim 24, wherein the muconic acid used has a ¹⁴C-to-¹²C isotope ratio in the range from 0.5×10⁻¹² to 5×10-¹².

33. The process according to claim 24, wherein the transition metal catalyst C includes at least one transition metal selected from Re, Fe, Ru, Co, Rh, Ir, Ni, Pd, Pt, Cu and Au.

34. The process according to claim 24, wherein the transition metal catalyst C comprises metallic nickel, metallic cobalt, metallic rhodium or a mixture of at least two of these metals.

35. The process according to claim 24, wherein the transition metal catalyst C is selected from Raney nickel, Raney cobalt, rhodium on a support material, and mixtures thereof.

36. The process according to claim 24, wherein at least a portion of the reaction mixture is withdrawn from the reaction zone, the reaction mixture withdrawn is subjected to a separation into an adipic acid-enriched fraction and an adipic acid-depleted fraction, and the adipic acid-depleted fraction is optionally at least partly recycled into the reaction zone.

37. The process according to claim 36, wherein the reaction mixture withdrawn from the reaction zone is subjected to a crystallization of the adipic acid and at least a portion of the mother liquor is recycled into the reaction zone.

38. A process for preparing adipic acid or at least one conversion product thereof, selected from hexane-1,6-diol, hexamethylenediamine and polyamide-6,6, in which muconic acid is hydrogenated with hydrogen in the presence of at least one heterogeneous transition metal catalyst C and of an aqueous liquid A in a reaction zone, wherein the muconic acid is at least partly insoluble in the liquid A under the hydrogenation conditions, and wherein
the liquid A has a water content in the range from 95 to 100% by weight, based on the total weight of the liquid A,
the transition metal catalyst C comprises metallic nickel, metallic cobalt, metallic rhodium or a mixture of at least two of these metals, and
at least a portion of the reaction mixture is withdrawn from the reaction zone, the reaction mixture withdrawn is subjected to a separation into an adipic acid-enriched fraction and an adipic acid-depleted fraction, the adipic acid-depleted fraction is at least partly recycled into the reaction zone.

39. The process according to claim 24, wherein the hydrogenation is conducted at a temperature in the range from 20° C. to 250° C.

40. The process according to claim 24, wherein the reaction is conducted at an absolute hydrogen pressure in the range from 1 to 300 bar.

41. The process according to claim 24, wherein the hydrogenation is conducted in n series-connected hydrogenation reactors, where n is an integer of at least two, and wherein the 1st to (n-1) th reactor has a stream from the reaction zone which is conducted within an external circuit and the hydrogenation in the nth reactor is conducted adiabatically.

42. A process for preparing hexane-1,6-diol, in which
a) muconic acid is subjected to a hydrogenation with hydrogen in the presence of an aqueous liquid A and in the presence of at least one transition metal catalyst C wherein the transition metal catalyst C includes at least one transition metal selected from the group consisting of Ru, Co, Rh, Ir, Ni, Fe, Pd, Pt, Cu and Au, to obtain adipic acid;
b) the adipic acid obtained in step a) is subjected to a reaction with hydrogen in the presence of at least one hydrogenation catalyst.

43. The process according to claim 42, wherein the hydrogenation catalyst used in step b), based on the total weight of the reduced catalyst, comprises at least 50% by weight of elements selected from rhenium, iron, ruthenium, cobalt, rhodium, iridium, nickel and copper.

44. A process for preparing hexamethylenediamine, in which
a) muconic acid is subjected to a hydrogenation with hydrogen in the presence of an aqueous liquid A and in the presence of at least one transition metal catalyst C as defined in claim 24, to obtain adipic acid,
b) the adipic acid obtained in step a) is subjected to a reaction with hydrogen in the presence of at least one hydrogenation catalyst to give hexane-1,6-diol,
c) the hexane-1,6-diol obtained in step b) is subjected to an amination with ammonia in the presence of an amination catalyst to obtain hexamethylenediamine.

45. A process for preparing polyamide-6,6, in which
a) muconic acid is subjected to a hydrogenation with hydrogen in the presence of an aqueous liquid A and in the presence of at least one transition metal catalyst C as defined in claim 24, to obtain adipic acid,
b) the adipic acid obtained in step a) is subjected to a reaction with hydrogen in the presence of at least one hydrogenation catalyst to give hexane-1,6-diol,
c) the hexane-1,6-diol obtained in step b) is subjected to an amination with ammonia in the presence of an amination catalyst to obtain hexamethylenediamine,
d) the hexamethylenediamine obtained in step c) is subjected to a polycondensation with adipic acid to obtain polyamide-6,6.

46. The process according to claim 45, wherein the adipic acid used in step d) is prepared at least partly by the process according to claim 24.
